(19) United States
(12) Patent Application Publication
(10) Pub. No.: US 2024/0060986 A1
(43) Pub. Date: Feb. 22, 2024

(54) METHOD FOR SELECTING PEPTIDE INHIBITORS BASED ON PROTEIN CYTOTOXICITY

(71) Applicant: Alexander Pisarchik, Halethorpe, MD (US)

(72) Inventor: Alexander Pisarchik, Halethorpe, MD (US)

(21) Appl. No.: 18/236,189

(22) Filed: Aug. 21, 2023

Related U.S. Application Data
(60) Provisional application No. 63/373,050, filed on Aug. 20, 2022.

(51) Int. Cl. G01N 33/68

(52) U.S. Cl. CPC G01N 33/6845

(57) ABSTRACT

A Method for selecting peptide inhibitors based on protein cytotoxicity by inserting a cytotoxic target protein and a library of peptide variants in a host cell, expressing the cytotoxic protein and peptide variants in the host cell, and identifying the peptide variants that block the cytotoxic target protein. The cytotoxic target protein is formed by synthesizing a cytotoxic protein gene and cloning the cytotoxic protein gene with an expression vector in a host cell. The library of peptide variants is formed by synthesizing a peptide library or building a peptide library using degenerate oligos and by cloning the peptide library with an expression vector in the host cell. Peptide variants are identified by identifying growing clones through growth on solid media or in a liquid culture. Both cyclic and linear peptides are produced. Intracellular biasing libraries of peptide variants produce inhibitor peptides with lower toxicity and higher stability compared to current methods.

### CROSS REFERENCE TO RELATED APPLICATION

This application claims priority to and the benefit of U.S. Provisional Patent Application No. 63/373,050, filed Aug. 20, 2022, which is incorporated by reference herein in its entirety for all purposes.

### FIELD OF THE DISCLOSURE

The method and system of this disclosure relates to the development of therapeutic peptides and, more particularly, to a method of generating peptide inhibitors and an intracellular cytotoxic target protein intracellularly and identifying peptides from the generated peptide inhibitors that inhibit the cytotoxicity of the cytotoxic target protein.

### BACKGROUND OF THE DISCLOSURE

Modern day drug discovery has focused on the development of small molecule therapeutics. While small molecules offer many advantages, such as economical manufacturing, lower complexity and better bioavailability as compared to legacy drugs, they can only target 2-5% of the proteome[1,2]. Biologic-based drugs have a larger binding surface and therefore a higher target specificity, allowing them to access targets that are beyond the reach of small molecules. However, most biologics are large molecules that cannot cross cell membranes, which restricts their use to extracellular targets. Peptide drugs, on the other hand, have advantages of both small molecule therapeutics and biologic drugs, but do not have many of their disadvantages. Like biologic-based drugs, peptides have a large binding surface to target leading to their higher specificity and fewer off-target effects[3,4]. Similar to small molecules, they are smaller, have lower immunogenicity [5,6] and higher bioavailability. Recent advances in cell penetrating peptide technology have enabled peptide drugs to be designed to access intracellular targets[7,8]. Peptide drugs can therefore achieve the level of bioavailability comparable to that of small molecule therapeutics and activity and safety of biologic-based drugs which makes them prime candidates of drug development for previously “undruggable” targets.

The development of therapeutic peptides commonly starts with a combinatorial biology approach that involves the generation of chemical or biosynthetic peptide libraries. Chemical peptide synthesis is a well-established method for developing peptide libraries[9,10]; however, the biosynthetic approach offers many advantages. One key advantage is the library size. Biosynthetic libraries can easily contain as many as 10⁹ peptides, while chemical synthesis is limited to approximately 10⁴ peptides. The most commonly used biosynthetic selection methods are phage display[11], yeast display[12] and RNA display[13,14]. All of these methods select peptides that bind to the target protein most tightly. However, a major limitation to these approaches is that the best binders may not be the best inhibitors of the target protein.

One way to solve this problem is to establish a link between binding and function by screening peptides intracellularly for their ability to attenuate or inhibit cellular processes. None of the existing cell-based assays has taken full advantage of this approach. Currently, the most promising in vivo peptide selection method, called split-intein circular ligation of peptides and proteins (SICLOPPS), is based on protein trans-splicing. This involves self-excision of an internal protein segment (intein) resulting in a cyclized polypeptide[15]. Typically, such libraries are screened in E. coli cells using bacterial two-hybrid system. Selection relies on disruption of a targeted protein-protein interaction (not function), detected through a reporter gene expression[16]. False positive clones often result due to fluctuations of gene expression, mutations in the regulatory sequences and mutations in the bacterial genome. Additionally, construct design for these peptide “processing” enzymes (inteins) is complex, they mostly work in a reduced environment[17], and are often slow[18].

### SUMMARY OF THE DISCLOSURE

This disclosure describes a method for selecting peptide inhibitors based on protein cytotoxicity which includes inserting a cytotoxic target protein and a library of peptide variants in a host cell, expressing the cytotoxic protein and peptide variants in the host cell; and identifying the peptide variants that block the cytotoxic target protein. The cytotoxic target protein is formed by synthesizing a cytotoxic protein gene and cloning the cytotoxic protein gene with an expression vector in a host cell. The library of peptide variants is formed by synthesizing a peptide library or building a peptide library using degenerate oligos and by cloning the peptide library with an expression vector in the host cell. The toxicity of the cytotoxic target protein is validated by expressing the cytotoxic protein in the host cell. The cytotoxic target protein and peptide are expressed, preferably, by adding an inducer to the host cell. The peptide variants are identified by identifying growing clones through growth on solid media or in a liquid culture and by identifying peptide sequences of the peptides that inhibit the cytotoxic target protein through sanger or Next Generation Sequencing. The in-vitro inhibitory potency of the peptides on the cytotoxic target protein is tested. If desired, peptide open reading frames may be inserted in a carrier protein (ubiquitin) gene.

An advantage of the method of this disclosure is a connection between binding and function.

Another advantage is screening for both cyclic and linear peptides.

Another advantage is a method that relies on the toxicity of a target protein in a host cell.

Another advantage is co-expression of a cytotoxic target protein and a library of peptide variants in a host cell.

Another advantage is intracellular biasing libraries of peptide variants producing inhibitor peptides with lower toxicity and higher stability.

### BRIEF DESCRIPTION OF THE DRAWINGS

FIG. 1A illustrates a condition in which intracellular peptide generation produces no peptides that inhibit the intracellular cytotoxic target protein.

FIG. 1B illustrates a condition in which intracellular peptide generation produces peptides that inhibit the intracellular cytotoxic target protein.

FIG. 2 illustrates the creation of a pMpro plasmid by amplifying M^{pro} and GST genes, wherein a pUbi-Mpro construct uses an arabinose-inducible promoter to expresses ubiquitin and M^{pro} as an operon.

FIG. 3A illustrates strains containing constructs with wild type protease do not grow on plates with arabinose while strains containing empty vector or mutant M^{pro} protease did grow.

FIG. 3B shows expressed and purified M^{pro} variants with mutations in the active site.

FIG. 4 shows a flow chart of the method of the peptide library construction of this disclosure.

### DETAILED DESCRIPTION OF THE DISCLOSURE

While the following description details the preferred embodiments of the method of this disclosure, it is to be understood that the system and method are not limited in their application to the details of arrangement of the parts or steps of the methods illustrated in the accompanying figures, since the system and method are capable of other embodiments and of being practiced in various ways.

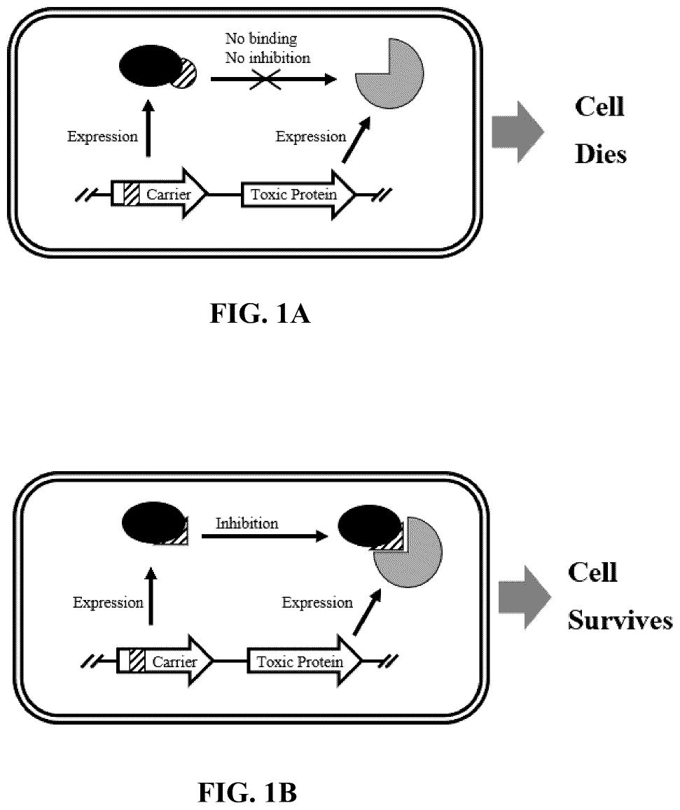

This disclosure describes a peptide selection method and system based on direct inhibition of a cytotoxic target protein (FIG. 1). Peptides mimic cyclization by insertion into a protein loop, thus avoiding the need for any processing enzymes (like inteins). This method and system give flexibility of screening both cyclic and linear peptides, which further increases the library size and improves the chances for identification of the optimal peptide inhibitor. Selections for a small pool of peptides (10⁶ variants) that consisted of cyclic and linear peptide inhibitors targeting main coronavirus protease (M^{pro}) were preformed and within five weeks, a peptide inhibitor with an IC₅₀ of 33 μM against M^{pro} was identified.

### EXAMPLE

### Constructs

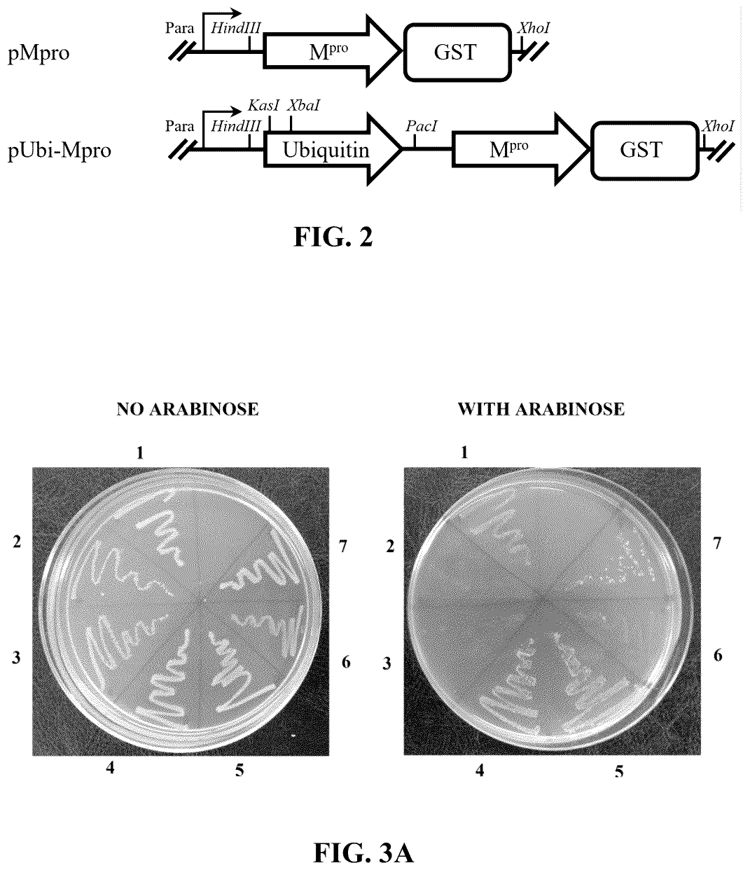

All genes were codon optimized, synthesized as gBlocks by IDT and cloned into the pBAD-HisA plasmid (Thermo Fisher Scientific). pBAD-HisA plasmid was amplified with primers P33 and P34 (Table 1) introducing HindIII and XhoI restriction sites. To create pMpro plasmid (FIG. 2) M^{pro} and GST genes were amplified. M^{pro} gene was amplified by primers T111 and T231. GST was amplified by primers P230 and P108 (Table 1) introducing XbaI and XhoI restriction sites. Each PCR reaction (20 μl) contained 20 ng of DNA template and 50 pmoles of each primer mixed with 10 μl of Pfu Ultra II Hotstart 2× Mastermix (Agilent). The PCR reaction (20 μl) was initially heated at 95° C. for 2.5 min followed by 30 cycles of denaturation at 94° C. for 15 sec., annealing at 55° C. for 15 sec. and extension at 72° C. for 6 min. Following amplification, the PCR fragments were gel-purified by the QIAGEN gel-band purification kit, mixed and amplified with primers T111 and T108 as described above. PCR fragments were gel-purified by the QIAGEN gel-band purification kit, digested with HindIII and XhoI restriction enzymes, purified again and ligated with pBAD plasmid (cut with HindIII and XhoI). The ligation reaction (20 μl) contained 2 μl of 10× ligation buffer, 100 ng of each fragment and 1 μl of T4 DNA ligase (NEB cat #M0202S). The reaction mix was incubated for 1 h at room temperature. Ligated fragments were transformed in 10G chemically competent cells (Lucigen) according to manufacturer's protocol. Transformed cells were plated on LB plates containing 50 μg/mL ampicillin and incubated overnight at 37° C. The insert was confirmed with colony PCR. This step involved resuspending a colony in 20 μl of sterile 0.9% sodium chloride solution. One μl of this solution was transferred to the PCR tube and amplified with Taq polymerase (New England Biolabs, cat #M0482S) and 30 pM of the flanking primers. Each PCR reaction (20 μl) was initially heated to 95° C. for 2.5 min followed by 30 cycles of denaturation at 94° C. for 20 seconds, annealed at 55° C. for 20 seconds, and extended at 72° C. for 1 min. Amplification products were visualized by agarose electrophoresis. Clones with the correct inserts were inoculated in culture tubes containing 5 ml of LB with the appropriate antibiotic and incubated overnight at 37° C. Constructs were then purified using the Monarch Plasmid miniprep kit (NEB) and sequenced.

TABLE 1
Peptide Inhibitors of M^{Pro} Protease

| Peptide | Peptide | IC50 | N = 4, |
|---|---|---|---|
| Name | Sequence | (μM) | P < 0.05 |
| M1 | GARQGLDEDLHRW | linear | 249 ± 47 |
| M5 | GATANAFLSGSGSRG | linear | 101 ± 17 |
| M5c | WRRWWRRRRTANAFLS | cyclic | 34 ± 8 |

Selected Peptide Sequences are Underlined

The pUbi-Mpro construct (FIG. 2.) uses an arabinose-inducible promoter to express ubiquitin and M^{pro} as an operon. A Shine-Dalgarno sequence is inserted between M^{pro} and Ubiquitin to ensure the expression of both genes. The ubiquitin gene was synthesized by IDT and amplified by primers T227 and T228 (Table 1). The amplified fragment was cut with HindIII and PacI restriction enzymes. PCR conditions are described above, with the exception that extension was performed for 1 minute. The M^{pro}-GST fusion was amplified with primers T229 and P108 (Table 1) and cut with PacI and XhoI restriction sites as described above. Following amplification, both PCR fragments were gel-purified using the QIAGEN gel-band purification kit. Then, both fragments were ligated with pBAD backbone (cut with HindIII and XhoI). The rest of the cloning procedures were performed as described above.

### M^{pro} Mutagenesis:

M^{pro} mutants were created by extension PCR in the pMpro construct. Primers used for this PCRs are shown in Table 1. To create each mutant, two fragments were amplified. Left fragment was amplified by primers P23 (Table 1) and the mutagenic reverse primer: T210 for M^{pro} mutant C12A; T212 for mutant H42A; T214 for mutant C146A and T216 for mutant R299A (Supplemental Table 2). The right fragment was amplified by the reverse primer P24 (Table 1) and mutagenic forward primer: T209 for M^{pro} mutant C12A; T211 for mutant H42A; T213 for mutant C146A and T215 for mutant R299A (Supplemental Table 2). Each PCR amplification reaction contained 30 pmol of each primer and 100 ng of DNA template. Amplifications were carried out using Pfu Ultra II Hotstart DNA polymerase (Agilent, cat #600850-51). The PCR reaction (20 μL) was initially heated at 95° C. for 2.5 min followed by 30 cycles of denaturation at 94° C. for 15 seconds, annealing at 55° C. for 15 seconds. and extension at 72° C. for 1 minute. Following amplification, the PCR fragment was gel-purified by the QIAGEN gel-band purification kit and mixed. These mixtures served as templates for the extension PCR by primers P23 and P24 to generate the full-length gene fragment. Fragments were gel-purified using QIAGEN gel-band purification kit, cut with HindIII and XhoI restriction enzymes and purified with the QIAGEN gel-band purification kit again. The final construct was ligated with pBAD backbone (also cut with HindIII and XhoI) by T4 DNA ligase (New England Biolabs) and transformed into the 10 G chemically competent cells (Lucigen, Cat #60107-2) according to manufacturer's protocol. Transformed cells were plated on LB plates containing 100 μg/ml carbenicillin and incubated overnight at 37° C. Individual colonies were sequenced using Genewiz company. Selected colonies were used to isolate plasmid DNA by Qiagen Miniprep kit and for expressing M^{pro} protein using GST tag.

### Purification of the M^{pro}-GST Fusions

Selected colonies were inoculated into culture tubes containing 4 ml containing 100 μg/ml carbenicillin and incubated overnight at 37° C. with vigorous shaking. Next morning, 1 ml of the night culture was added to 100 ml of LB media with 100 μg/ml carbenicillin in 0.5 L flask and incubated it at 37° C. with shaking until culture's OD600 reached 0.4 at which point arabinose was added to the final concentration of 0.04%. Cultures were incubated 16 h at 30° C. with shaking and centrifugated in the Eppendorf centrifuge 5810R at maximum speed and frozen at −80° C. When needed, cell pellets were removed from the freezer, incubated at room temperature and lysed with 3 ml of BPER protein lysis reagent (ThermoFischer). Peptide-GST fusions were purified using glutathione agarose (ThermoScientific cat #16100) as described by the manufacturer.

### Construction of Peptide Libraries

Random and candidate peptide libraries of the M^{pro}-inhibitor peptides were cloned into the first loop of ubiquitin (in the pUbi-M^{pro} constructs), which was shown previously to be tolerant to insertions and deletions[19].

The random library was built with 14 NNK codons and amplified as two fragments which were united by ligation. The first fragment was amplified with flanking forward primer P23 (Table 1) and the reverse primer T232 (Supplemental Table 2). The second fragment was amplified with primer T233 (Supplemental Table 2) and the reverse primer P24 (Table 1). The PCR reaction (20 μL) was initially heated at 95° C. for 2.5 min followed by 30 cycles of denaturation at 94° C. for 15 sec., annealing at 55° C. for 15 sec. and extension at 72° C. for 40 sec. Following amplification, PCR fragments were gel-purified by the QIAGEN gel-band purification kit and mixed and ligated with T4 DNA ligase. The ligation reaction contained 20 μl of 10×ligation buffer, 100 ng of fragment mix, 0.5 μl of 100 mM ATP, 1 μl of T4 DNA ligase (NEB cat #M0202S) and 1 μl of T4 polynucleotide kinase. The reaction mix was incubated at room temperature and used as a template for PCR with flanking primers P276 and P277 (Table 1) as described above. The PCR fragment was gel-purified by the QIAGEN gel-band purification kit and cut with KasI and XbaI restriction enzymes, purified by QIAGEN kit and ligated with pUbi_Mpro also digested by KasI and XbaI. Ligated fragments were transformed in 10 G electrocompetent cells according to the recommendations of Bio-Rad. Transformed cells were plated on LB plates containing 50 μg/mL ampicillin and incubated overnight at 37° C. Individual clones were sequenced verified by GeneWiz.

The M^{pro} candidate library was based on sequences recognized by M^{pro22}. These sequences were mutagenized by degenerate synthetic oligonucleotides. Library size was controlled by targeting mutations to one position in each codon with only the first or the second codon position being changed. Library construction was done as described above with the following differences: the first fragment was amplified with flanking forward primer P23 (Table 1) and one of the reverse primers (Supplemental Table 2, primers T234 through T255). The right fragment was amplified with primer T233 (Table 1) and one of the forward primers (Supplementary Table 2, primers T256 through T277). Following amplification, PCR fragments were gel-purified and mixed and ligated with T4 DNA ligase. The full-length PCR band was amplified with the flanking primers P276 and P277 (Table 1), digested with KasI and XbaI restriction enzymes and ligated with pUbi-Mpro as described above.

### Selection of Libraries

Libraries were constructed as described above and desalted by dialysis as follows: 20 ml of Ultrapure water was poured into Petri dishes. 0.025 μM VSWP Membrane Filters were placed on top of the water. 10 μl drops of the DNA were pipetted on filters and incubated for 30 min. Following incubation DNA concentration was measured. Libraries were transformed into 10 G strain of E. coli using electroporation. To make electrocompetent cells one colony from a freshly streaked plate of the E. coli was inoculated in 5 ml of LB and grown overnight at 37° C. with shaking at 250 rpm overnight. Then 2 ml of the overnight E. coli culture was transferred to two 500 ml flasks containing 250 ml LB media each. Flasks were shaken at 250 rpm and incubated at 37° C. until OD600 reached 0.4. Cultures were centrifugated at 4000×g at 4° C. for 15 min. The supernatant was removed, cells were resuspended in 100 ml of sterile ice-cold water and centrifugated at 4000×g at 4° C. for 15 min. This step was repeated a second time and then the cells were resuspended in 50 ml of ice-cold water and centrifugated at 4000×g at 4° C. for 15 min. After repeating the step for the third time, cells were resuspended in 20 ml of sterile ice-cold 10% glycerol and centrifugated at 3000×g at 4° C. for 15 min. Supernatant was discarded and cells were resuspended in 1 ml of 10% glycerol. Micropulser electroporator (Bio-Rad) and 0.1 mm cuvettes were used for electroporation according to the manufacturer's recommendations. Following electroporation, 1 ml of SOC media was added to each transformation. Tubes were shaken at 37° C. and 250 rpm for 1 h and added to the 500 ml flasks containing 250 ml LB media with 100 μg/ml carbenicillin and 0.4% arabinose and incubated overnight at 37° C. with shaking.

Following selection, libraries were re-cloned into the original vector pUbi-M^{pro} to remove false positive sequences (clones that grow in the media but didn't express M^{pro} protease). For this purpose, 3 ml of cell culture was used for plasmid isolation using Qiagen Miniprep Kit. Libraries were amplified from plasmid populations using primers P276 and P277 (Table 1). Each PCR reaction (20 μl) contained 20 ng of DNA template and 50 pmoles of each primer mixed with 10 μl of Pfu Ultra II Hotstart 2×Mastermix (Agilent). The PCR reaction (20 μL) was initially heated to 95° C. for 2.5 min followed by 30 cycles of denaturation at 94° C. for 15 sec., annealing at 55° C. for 15 sec. and extension was at 72° C. for 40 sec. Following amplification, the PCR fragment was gel-purified by the QIAGEN gel-band purification kit and used for Next Generation Sequencing (NGS), re-cloning in the original vector and electroporated into 10 G cells as described above.

### Sequence Analysis

Sequences of individual clones were analyzed by Next Generation Sequencing (NGS) and Sanger Sequencing. NGS was performed by submitting PCR reactions from each selection cycle to GeneWiz for Amplicon-EZ service. Libraries were also streaked on Petri dishes with LB agar containing appropriate antibiotics and incubated overnight at 37° C. Between 10 and 20 bacterial colonies were submitted for Sanger sequencing to GeneWiz. The sequences of the most abundant peptides identified by NGS and confirmed by Sanger sequencing, were selected for further testing.

### Inhibition of the M^{pro} Protease Activity by the Peptides In Vitro

Peptides were synthesized by Elim Biopharmaceuticals at 95% purity. Inhibitory activity of these peptides on M^{pro} was tested using 3CL Protease Kit from BPS Bioscience (Catalog #78042-1) according to the manufacturer's recommendations. Briefly, 30 μl of 3CL Protease enzyme solution (0.05 ng/ul) was mixed with 10 μl of peptides at different concentrations and preincubated for 30 min at room temperature. Following preincubation, 10 μl of 200 μM 3CL Protease substrate was added and incubated for 4 h at room temperature. The fluorescence intensity was measured in a microtiter plate-reading fluorimeter with excitation at 360 nm and emission at 460 nm.

### Selection System Results

The selection system described here relies on the toxicity of a target protein to its host (FIG. 1). A peptide variant is co-expressed with the cytotoxic target protein in the host cell. The host cell only survives if a peptide variant binds to the cytotoxic target protein and neutralizes its cytotoxicity. We chose the main protease (M^{pro})[20] of SARS-CoV2 virus as an example. M^{pro} plays a central role in the virus life cycle[21]. It processes viral polyproteins and controls the replicase complex activity[22] which makes it a very attractive target for drug development.

To confirm cytotoxicity of M^{pro} wild type and mutant M^{pro} proteases were expressed in 10 G strain of E. coli. Four M^{pro} mutants were tested. Mutations H42A and C146A were located in the active site and were expected to inactivate protease completely. Mutations G12A and R299A were previously shown to be involved in protease dimerization[20]. They decreased enzyme activity, but some residual enzymatic activity was still possible.

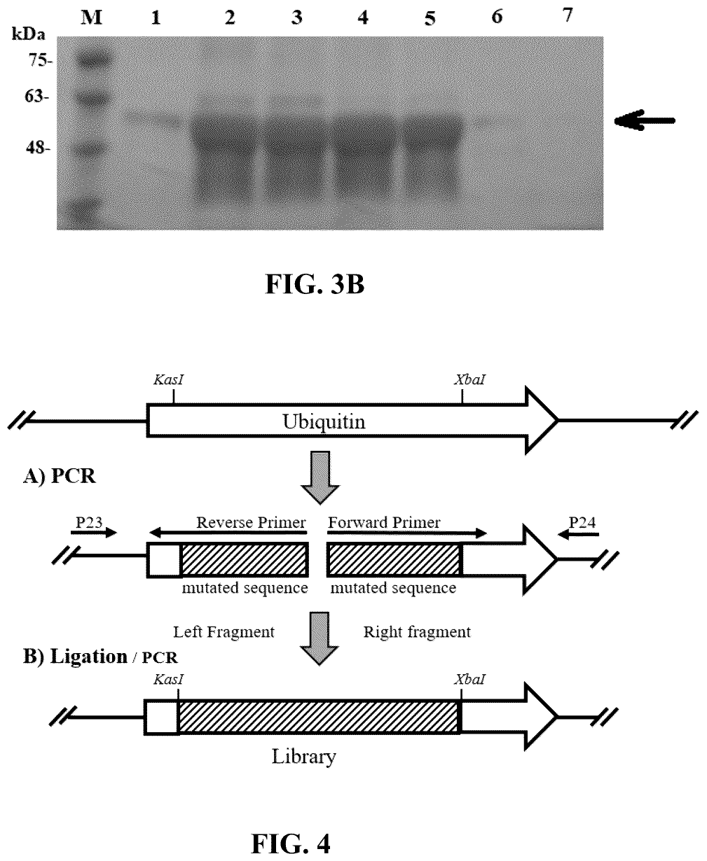

E. coli with expression constructs containing wild type and mutant M^{pro} protease genes were streaked on plates with and without 0.4% arabinose and incubated at 37° C. overnight. FIG. 3A shows strains containing constructs with wild type protease did not grow on plates with arabinose (FIG. 3A, sections 2 and 3) while strains containing empty vector or mutant M^{pro} protease did grow (FIG. 3A, sections 1, 4-7). Mutations located in the protease's active site had the least effect on bacterial growth (mutants H42A and C146A, FIG. 3A, sections 4 and 5). Mutations affecting protease dimerization slowed down bacterial growth but didn't stop it completely (Mutants G12A and R299A, FIG. 3A, sections 6 and 7).

Purification of the M^{pro} variants using standard purification on GST resin showed similar results. While any protein from the strain expressing wild type M^{pro} (FIG. 3B, lane 7) could not be purified, both M^{pro} variants with mutations in the active site were easily expressed and purified (FIG. 3B, lanes 2-5). Mutations targeting M^{pro} residues involved in dimerization (G12A and R299A, FIG. 3B, lanes 1 and 6) showed improvement in protein yield but the improvement was significantly lower than one observed for the active site mutants. The results show that expression of the wild type enzyme inhibits bacterial growth, while expression of the mutants lacking enzymatic activity does not, indicating that toxicity of the M^{pro} protease is caused by its enzymatic activity.

### Library Results

Ubiquitin was used as a carrier protein. The peptide libraries were inserted into the first loop of ubiquitin because ubiquitin is a small protein (8.6 kD) which is stable in E. coli and has previously been used to express proteins and peptides[23,24]. The first loop was chosen as a site for library insertion because loops are generally tolerant to insertions and deletions and the first loop was previously used for insertions[19].

Ubiquitin was co-expressed with M^{pro} from the same expression construct pUbi-Mpro which is shown in FIG. 2. In this construct Ubiquitin and M^{pro} genes are arranged in an operon fashion under the control of the arabinose-inducible promoter. A Shine-Dalgarno sequence is inserted between M^{pro} and Ubiquitin to ensure the expression of both genes.

The method Library construction is presented in FIG. 4. The first peptide library was random, built with 14 degenerate codons, resulting in up to 1.6×10¹⁸ variants. The second library was based on published sequences [25] recognized by M^{pro}, and contained approximately 2×10⁹ variants. These libraries were inserted into the first loop of ubiquitin. Variant sequences with no stop codons or frame-shifts were fully integrated into the loop of the full-length ubiquitin protein and served as a model of cyclic peptides. Variants with stop codons were expected to produce linear peptides attached to the first beta strand of ubiquitin.

### Peptide Evolution Results

Both peptide libraries were cloned in the pUbi-Mpro construct (FIG. 2) and taken through five rounds of selection in E. coli. 1 million clones at each round were screened. To weed out false positives that may result from frame-shifts, deletions of M^{pro} and somatic mutations libraries were re-cloned into the original vector (pUbi-Mpro) after each round of selection. The fifth round of selection generated several sequences that were significantly overrepresented in the population. The 11 most abundant peptides were chosen for further testing. Seven of these peptides were linear. 4 of these peptides were fully integrated in the loop of the carrier protein and, therefore, were cyclic (Supplemental Table 3). The most abundant peptides were synthesized in a linear form and tested in an in vitro M^{pro} activity assay. Out of 11 peptides tested, 7 did not have any effect on protease activity (false positives). The other 4 peptides inhibited the M^{pro} with IC50s ranging from 100 μM to 1.2 mM (Supplementary Table 3). The two best peptides were M1 (RQGLDEDLHRW) and M5 (TANAFLS). Their IC50s were 249 and 101 μM, respectively (Supplemental Table 4). Peptide M1 originated from the random library and peptide M5 originated from the library based on the published sequences that are recognized by M^{pro}. To be consistent with the structure in the original screen, peptide M5 was also synthesized in a cyclic form (peptide M5c) and fused to a custom cell penetration sequence (WRRWWRRRR) to improve its stability and intracellular transport. Cyclization improved the IC50 of M5 peptide significantly from 101 to 33 μM (Supplemental Table 4).

A significant advantage of the current display technologies (eg. Phage display, RNA display, yeast display) is a connection between binding and function. That means that a peptide binding to the target protein will inhibit its enzymatic activity or disrupt a protein-protein interaction. The selection of a peptide is based on the cytotoxicity of the target protein. Other in vivo selection methods have relied on the toxicity of an enzyme's (target protein) substrate[26], products of the enzymatic reaction[27], a particular intermediate[28], or resistance to inhibitors[29]. The peptide selection approach of this disclosure is the first peptide selection method to capitalize on the cytotoxicity of the target protein itself.

The problem of protein toxicity is widespread in the field of protein expression. Usually, it is a problem that has to be minimized. The method of this disclosure leverages this “problem” by screening peptides for their ability to neutralize the cytotoxicity of the target protein. The method involves co-expression of the cytotoxic target protein and a library of peptide variants in a host cell. Host cells survive only when a particular peptide variant inhibits the cytotoxic protein (FIG. 1).

To demonstrate the power of this technology, the coronavirus M^{pro} protease was targeted. M^{pro} highly conserved among various coronaviruses and plays a pivotal role in the life cycles[21] of the coronaviruses. Mutations in M^{pro} are often lethal to the virus which is why drugs targeting the M^{pro} enzyme have the potential to significantly reduce the risk of mutation-mediated drug resistance and display broad-spectrum antiviral activity[20]. To date, no M^{pro} targeted antiviral drug has been developed. Repurposing of antiviral drugs for other viruses[30,31] have not proved effective. Drug development approaches based on converting peptides into peptidomimetics are also very challenging, because side chain modifications often abolish inhibitor activity[32] and result in off-target effects[33] and toxicity[34]. The method of this disclosure avoids these pitfalls because peptides are selected intracellularly, biasing libraries towards candidates with lower toxicity and higher stability.

The two best peptides (M1 and M5) generated by the peptide selection method and system disclosed herein showed inhibitory activity in the low micromolar range in an in vitro assay (Table 1), demonstrating the utility of this peptide selection approach. Peptide M1 was selected from the random library and peptide M5 from the candidate-based library. This observation demonstrates that this approach can identify inhibitors without prior knowledge of their ligands and can improve the inhibitory activity of known ligands. Furthermore, peptide M5 is fully integrated in the first loop of the carrier protein (ubiquitin) which gives peptide M5 a cyclic structure. Consistent with this observation, when peptide M5 was cyclized, its IC50 improved significantly from 101 to 33 μM (Table 1, peptide M5c) which confirms that this technology is useful for screening both linear and cyclic peptides. This method and system of selecting peptide inhibitors base on protein cytotoxicity is able to rapidly identify (in a few weeks) potent peptide inhibitors with low μM activity (Supplemental Table 4).

The foregoing description illustrates and describes the method and system of the disclosure. Additionally, the disclosure shows and describes only the preferred embodiments, but it is to be understood that the preferred embodiments are capable of being formed in various other combinations, modifications, and environments and are capable of changes or modifications within the scope of the invention concepts as expressed herein, commensurate with the above teachings and/or the skill or knowledge of the relevant art. The embodiments described herein above are further intended to explain the best modes known by applicant and to enable others skilled in the art to utilize the disclosure in such, or other, embodiments and with the various modifications required by the particular application or uses thereof. Accordingly, the description is not intended to limit the invention to the form disclosed herein. Also, it is intended that the appended claims be construed to include alternative embodiments. It will be further understood that various changes in the details, materials, and arrangements of the parts which have been described and illustrated above to explain the nature of this invention may be made by those skilled in the art without departing from the principle and scope of the invention as recited in the following claims.

SUPPLEMENTARY TABLE 2
Primers used for plasmid construction and PCR

| Name | Sequence |
|---|---|
| P23 | CCGCGAATGGTGAGATTGAGAA |
| P24 | ACGCAAAAAGGCCATCCGTCAG |
| P33 | aactaagcttTTCCTCCTGTTAGCCCAAAAAAC |
| P34 | aatactcgagGCTGTTTTGGCGGATGAGAGAA |
| P108 | aatactcgagTTATTTTGGAGGATGGTCGCCACCA |
| P209 | aataaagcttATGtctagaGGTTCTGGCTCAGGTTCTTCC |
| P276 | ATGCAAATCTTCGTCAAGACCTTG |
| P277 | GCTCCACTTCCAGTGTGATAGTC |
| T111 | GGAAaagcttATGTCGGGATTCCGTAAGATG |
| T112 | ataatctagaACCTTGGAAGTAAAGGTTTTCac |
| T227 | TTAAaagcttATGCAAATCTTCGTCAAGACCTTG |
| T228 | CCTTAttaattaaTTATCCACCGCGAAGACGTAAAAC |
| T229 | TTAAttaattaaTAAGGAGGTacgcgtATGTCGGGAT |
| | TCCGTAAGATGG |
| T230 | CTTTACTTCCAAGGTtctGGTTCTGGCTCAGGTTCTTCC |
| T231 | ACCTGAGCCAGAACCagaACCTTGGAAGTAAAGGTTTT |
| | Cac |
| T333 | accagaacctgagccagaaccagaCTGAAACGTCACACC |
| | TGAACATTG |
| T334 | tctggttctggctcaggttctggtGCGAGCTTGGTTAAG |
| | AAAGATATG |
| T335 | CTAATAActcgagTTACGTCGCTTTTTCCGGCACAT |
| T336 | atgcttTTCCTCCTGTTAttaattaaTTATCCACCG |
| | CGAAGACGTAAAAC |
| T337 | ttaattaaTAACAGGAGGAAaagcatATGTCGGGAT |
| | TCCGTAAGATGG |

SUPPLEMENTAL TABLE 3
Mutagenic Primers

| Name | Sequence |
|---|---|
| P23 | CCGCGAATGGTGAGATTGAGAA |
| P24 | ACGCAAAAAGGCCATCCGTCAG |
| T209 | ATGGCATTTCCGAGCgcaAAAGTTGAGGGATGCATG |
| T210 | GCATCCCTCAACTTTtgcGCTCGGAAATGCCATCTT |
| T211 | GTGTACTGTCCACGTgcaGTCATCTGTACTAGCGAA |
| T212 | GCTAGTACAGATGACtgcACGTGGACAGTACACAAC |
| T213 | TTCTTAAATGGCAGTgcaGGTTCAGTTGGATTTAAT |
| T214 | AAATCCAACTGAACCtgcACTGCCATTTAAGAATGA |
| T215 | CCATTCGATGTTGTCgcaCAATGTTCAGGTGTGACGT |
| T216 | CACACCTGAACATTGtgcGACAACATCGAATGGAGT |
| T232 | MNNMNNMNNMNNMNNMNNMNNMNNgccggcACCACGCCGACGTTGACGACGCTT |
| T233 | NNKNNKNNKNNKNNKNNKNNKNNKggtagtggctctagaggtGGTAAG |
| T234 | ACNTTNAANCGNAANACNTGNACNgccggcACCACGCCGACGTTGAC |
| T235 | CGNTTNCANCANTGNTGNGGNAANgccggcACCACGCCGACGTTGAC |
| T236 | AGNCTNAANCGNGGNCANCTNAANgccggcACCACGCCGACGTTGAC |
| T237 | TGNCTNCANGGNCGNGCNGTNATNgccggcACCACGCCGACGTTGAC |
| T238 | ATNCTNCANTTNAANGGNGCNATNgccggcACCACGCCGACGTTGAC |
| T239 | TGNCTNAANGCNCANCGNTGNGGNgccggcACCACGCCGACGTTGAC |
| T240 | GCNTTNCANTANCGNTTNACNTANgccggcACCACGCCGACGTTGAC |
| T241 | CGNCTNTANGANAGNGTNAGNAGNgccggcACCACGCCGACGTTGAC |
| T242 | TGNCTNCANCGNCGNAANGTNGCNgccggcACCACGCCGACGTTGAC |
| T243 | GGNCTNCANACNAGNAANAGNGTNgccggcACCACGCCGACGTTGAC |
| T244 | TGNCTNCANTTNAGNGTNAANTGNgccggcACCACGCCGACGTTGAC |
| T245 | ANCTNGANACNTANCANCTNAANAgccggcACCACGCCGACGTTGAC |
| T246 | CNATNGCNGCNCTNCTNAGNTANTgccggcACCACGCCGACGTTGAC |
| T247 | ANACNGANCCNTGNCCNCCNTANTgccggcACCACGCCGACGTTGAC |
| T248 | TNCCNGCNGGNTCNCGNGGNTANCgccggcACCACGCCGACGTTGAC |
| T249 | ANTCNGCNGTNTANCGNCGNTANTgccggcACCACGCCGACGTTGAC |
| T250 | TNCCNGANGGNGCNCCNTTNCGNCgccggcACCACGCCGACGTTGAC |
| T251 | GNTTNGCNTTNACNGTNCANGTNAgccggcACCACGCCGACGTTGAC |
| T252 | CNCCNGTNAGNCANTGNGANGANTgccggcACCACGCCGACGTTGAC |
| T253 | TNCCNGCNACNTCNCANCGNTGNGgccggcACCACGCCGACGTTGAC |
| T254 | GNACNGCNAANGANTANAANTGNTgccggcACCACGCCGACGTTGAC |
| T255 | TNCCNGCNATNTANGGNAANATNTgccggcACCACGCCGACGTTGAC |
| T256 | NAANTCNAANAGNTTNTGNAGggtagtggctctagaggtGGTAAG |
| T257 | NGTNTTNGTNAGNTGNCANTCggtagtggctctagaggtGGTAAG |
| T258 | NAANTGNCGNATNTTNAGNGTggtagtggctctagaggtGGTAAG |
| T259 | NTTNCGNGCNAGNTTNCANCGggtagtggctctagaggtGGTAAG |
| T260 | NATNAGNTANCANCCNTGNCAggtagtggctctagaggtGGTAAG |
| T261 | NGANATNCGNCCNAGNTTNCTggtagtggctctagaggtGGTAAG |
| T262 | NCANATNCGNCTNCANTTNTGggtagtggctctagaggtGGTAAG |
| T263 | NTTNGANCGNGCNTCNTANGCggtagtggctctagaggtGGTAAG |
| T264 | NAANACNTTNCTNGGNTTNTCggtagtggctctagaggtGGTAAG |
| T265 | NTGNAANACNTTNCGNACNATggtagtggctctagaggtGGTAAG |
| T266 | NCTNAANCANGGNAANCTNGTggtagtggctctagaggtGGTAAG |
| T267 | ANATNCANAANGANTGNGANGggtagtggctctagaggtGGTAAG |
| T268 | GNTTNTCNTANGANGGNATNCggtagtggctctagaggtGGTAAG |
| T269 | ANAANGTNGGNTGNTANGTNTggtagtggctctagaggtGGTAAG |
| T270 | ANTGNGANCGNGTNTTNATNGggtagtggctctagaggtGGTAAG |
| T271 | ANTGNGTNATNACNCGNGGNAggtagtggctctagaggtGGTAAG |
| T272 | GNAANTGNGANCGNGGNTCNTggtagtggctctagaggtGGTAAG |
| T273 | GNAGNTGNGTNTANATNTTNGggtagtggctctagaggtGGTAAG |
| T274 | GNTGNAGNGTNCGNCTNATNCggtagtggctctagaggtGGTAAG |
| T275 | GNAANCGNTANTGNGCNTTNCggtagtggctctagaggtGGTAAG |
| T276 | TNGGNAANCGNTGNGTNCANTggtagtggctctagaggtGGTAAG |
| T277 | TNTCNAGNATNGCNACNTGNTggtagtggctctagaggtGGTAAG |
| T343 | ggtagtggctctagaggtGGTAAG |
| T344 | NCANCANACNCTNTCNTTNCTggtagtggctctagaggtGGTAAG |
| T345 | NCANACNCTNTCNTTNCTggtagtggctctagaggtGGTAAG |
| T346 | NACNCTNTCNTTNCTggtagtggctctagaggtGGTAAG |
| T347 | NCTNTCNTTNCTggtagtggctctagaggtGGTAAG |
| T348 | NTCNTTNCTggtagtggctctagaggtGGTAAG |
| T349 | NTTNCTggtagtggctctagaggtGGTAAG |
| T350 | NCTggtagtggctctagaggtGGTAAG |
| T351 | ANAGNAANCGNTTNCCNTTNTggtagtggctctagaggtGGTAAG |
| T352 | GNAANCGNTTNCCNTTNTggtagtggctctagaggtGGTAAG |
| T353 | ANCGNTTNCCNTTNTggtagtggctctagaggtGGTAAG |
| T354 | GNTTNCCNTTNTggtagtggctctagaggtGGTAAG |
| T355 | TNCCNTTNTggtagtggctctagaggtGGTAAG |
| T356 | CNTTNTggtagtggctctagaggtGGTAAG |
| T357 | TNTggtagtggctctagaggtGGTAAG |
| IT358 | NCAGNANACGNTNTCCNTNCTggtagtggctctagaggtGGTAAG |
| T359 | GNANACGNTNTCCNTNCTggtagtggctctagaggtGGTAAG |
| T360 | NACGNTNTCCNTNCTggtagtggctctagaggtGGTAAG |
| T361 | GNTNTCCNTNCTggtagtggctctagaggtGGTAAG |
| T362 | NTCCNTNCTggtagtggctctagaggtGGTAAG |
| T363 | CNTNCTggtagtggctctagaggtGGTAAG |
| T364 | ANANCAANCNCTTNCNTTTNTggtagtggctctagaggtGGTAAG |
| T365 | NCAANCNCTTNCNTTTNTggtagtggctctagaggtGGTAAG |
| T366 | ANCNCTTNCNTTTNTggtagtggctctagaggtGGTAAG |
| T367 | NCTTNCNTTTNTggtagtggctctagaggtGGTAAG |
| T368 | TNCNTTTNTggtagtggctctagaggtGGTAAG |
| T369 | NTTTNTggtagtggctctagaggtGGTAAG |
| T370 | gccggcACCACGCCGACGTTGAC |
| T371 | AGNAANGANAGNGTNTGNTGNgccggcACCACGCCGACGTTGAC |
| T372 | AANGANAGNGTNTGNTGNgccggcACCACGCCGACGTTGAC |
| T373 | GANAGNGTNTGNTGNgccggcACCACGCCGACGTTGAC |
| T374 | AGNGTNTGNTGNgccggcACCACGCCGACGTTGAC |
| T375 | GTNTGNTGNgccggcACCACGCCGACGTTGAC |
| T376 | TGNTGNgccggcACCACGCCGACGTTGAC |
| T377 | TGNgccggcACCACGCCGACGTTGAC |
| T378 | ANAANGGNAANCGNTTNCTNTgccggcACCACGCCGACGTTGAC |
| T379 | ANGGNAANCGNTTNCTNTgccggcACCACGCCGACGTTGAC |
| T380 | GNAANCGNTTNCTNTgccggcACCACGCCGACGTTGAC |
| T381 | ANCGNTTNCTNTgccggcACCACGCCGACGTTGAC |
| T382 | GNTTNCTNTgccggcACCACGCCGACGTTGAC |
| T383 | TNCTNTgccggcACCACGCCGACGTTGAC |
| T384 | TNTgccggcACCACGCCGACGTTGAC |
| T385 | AGNANGGANANCGTNTNCTGNgccggcACCACGCCGACGTTGAC |
| T386 | ANGGANANCGTNTNCTGNgccggcACCACGCCGACGTTGAC |
| T387 | GANANCGTNTNCTGNgccggcACCACGCCGACGTTGAC |
| T388 | ANCGTNTNCTGNgccggcACCACGCCGACGTTGAC |
| T389 | GTNTNCTGNgccggcACCACGCCGACGTTGAC |
| T390 | TNCTGNgccggcACCACGCCGACGTTGAC |
| T391 | ANAAANGNAAGNGNTTGNTNTgccggcACCACGCCGACGTTGAC |
| T392 | AANGNAAGNGNTTGNTNTgccggcACCACGCCGACGTTGAC |
| T393 | GNAAGNGNTTGNTNTgccggcACCACGCCGACGTTGAC |
| IT394 | AGNGNTTGNTNTgccggcACCACGCCGACGTTGAC |
| T395 | GNTTGNTNTgccggcACCACGCCGACGTTGAC |
| T396 | TGNTNTgccggcACCACGCCGACGTTGAC |
| T397 | NGTNAANGTNTGNACNAGNATNTGNATNGTNGGTAAggtagtggctctagaggtGGTAAG |
| T398 | NGTNTGNACNAGNATNTGNATNGTNGGTAAggtagtggctctagaggtGGTAAG |
| T399 | NACNAGNATNTGNATNGTNGGTAAggtagtggctctagaggtGGTAAG |
| T400 | NATNTGNATNGTNGGTAAggtagtggctctagaggtGGTAAG |
| T401 | NATNGTNGGTAAggtagtggctctagaggtGGTAAG |
| T402 | NGGTAAggtagtggctctagaggtGGTAAG |
| T403 | CNTCNAGNTCNGGNCGNGGNTCNGCNTCNTTNGTAAggtagtggctctagaggtGGTAAG |
| T404 | CNAGNTCNGGNCGNGGNTCNGCNTCNTTNGTAAggtagtggctctagaggtGGTAAG |
| T405 | CNGGNCGNGGNTCNGCNTCNTTNGTAAggtagtggctctagaggtGGTAAG |
| IT406 | GNGGNTCNGCNTCNTTNGTAAggtagtggctctagaggtGGTAAG |
| T407 | CNGCNTCNTTNGTAAggtagtggctctagaggtGGTAAG |
| T408 | CNTTNGTAAggtagtggctctagaggtGGTAAG |
| T409 | TAAggtagtggctctagaggtGGTAAG |
| T410 | NGTCNANGTCNGNACGNGNATCNGNATCNTNGGTAAggtagtggctctagaggtGGTAAG |
| T411 | NGTCNGNACGNGNATCNGNATCNTNGGTAAggtagtggctctagaggtGGTAAG |
| T412 | NACGNGNATCNGNATCNTNGGTAAggtagtggctctagaggtGGTAAG |
| T413 | NATCNGNATCNTNGGTAAggtagtggctctagaggtGGTAAG |
| T414 | NATCNTNGGTAAggtagtggctctagaggtGGTAAG |
| T415 | TNGTAAggtagtggctctagaggtGGTAAG |
| T416 | CNTNAAGNTNTGGNCNAGGNTNTGCNTNGTTNGTAAggtagtggctctagaggtGGTAAG |
| T417 | NAAGNTNTGGNCNAGGNTNTGCNTNGTTNGTAAggtagtggctctagaggtGGTAAG |
| T418 | NTGGNCNAGGNTNTGCNTNGTTNGTAAggtagtggctctagaggtGGTAAG |
| IT419 | NAGGNTNTGCNTNGTTNGTAAggtagtggctctagaggtGGTAAG |
| T420 | NTGCNTNGTTNGTAAggtagtggctctagaggtGGTAAG |
| T421 | NGTTNGTAAggtagtggctctagaggtGGTAAG |
| T422 | TTACCNACNATNCANATNCTNGINCANACNTTNACNgccggcACCACGCCGACGTTGAC |
| T423 | ACNATNCANATNCTNGINCANACNTTNACNgccggcACCACGCCGACGTTGAC |
| T424 | CANATNCTNGTNCANACNTTNACNgccggcACCACGCCGACGTTGAC |
| T425 | CTNGTNCANACNTTNACNgccggcACCACGCCGACGTTGAC |
| T426 | CANACNTTNACNgccggcACCACGCCGACGTTGAC |
| T427 | TTNACNgccggcACCACGCCGACGTTGAC |
| IT428 | CNAANGANGCNGANCCNCGNCCNGANCTNGANGgccggcACCACGCCGACGTTGAC |
| T429 | ANGCNGANCCNCGNCCNGANCTNGANGgccggcACCACGCCGACGTTGAC |
| T430 | ANCCNCGNCCNGANCTNGANGgccggcACCACGCCGACGTTGAC |
| T431 | GNCCNGANCTNGANGgccggcACCACGCCGACGTTGAC |
| T432 | ANCTNGANGgccggcACCACGCCGACGTTGAC |
| T433 | ANGgccggcACCACGCCGACGTTGAC |
| T434 | ANGATNCNGATNCNCGTNCNGACNTNGACNgccggcACCACGCCGACGTTGAC |
| T435 | CNGATNCNCGTNCNGACNTNGACNgccggcACCACGCCGACGTTGAC |
| T436 | CNCGTNCNGACNTNGACNgccggcACCACGCCGACGTTGAC |
| T437 | CNGACNTNGACNgccggcACCACGCCGACGTTGAC |
| T438 | TNGACNgccggcACCACGCCGACGTTGAC |
| T439 | CNAACNANGCANANCCTNGNCCANANCTTNANGgccggcACCACGCCGACGTTGAC |
| T440 | ANGCANANCCTNGNCCANANCTTNANGgccggcACCACGCCGACGTTGAC |
| T441 | ANCCTNGNCCANANCTTNANGgccggcACCACGCCGACGTTGAC |
| T442 | GNCCANANCTTNANGgccggcACCACGCCGACGTTGAC |
| T443 | ANCTTNANGgccggcACCACGCCGACGTTGAC |
| T444 | ANGgccggcACCACGCCGACGTTGAC |

SUPPLEMENTAL TABLE 4
M^{Pro} Inhibition Assay of the peptides from
the first 5 rounds of selection

| | | Conformation | |
|---|---|---|---|
| Peptide | Peptide | in the | IC50 |
| Name | Sequence | library | (μM) |
| M1 | gaRQGLDEDLHRW | Linear | 250 |
| M2 | gaAKAHPQANV | Linear | not |
| | | | detected |
| M3 | gaRQDLDYQRRR | Linear | 750 |
| | GAGISSTLVQSRK | | |
| M4 | gaHCTFKLKDRKW | Cyclic | not |
| | VARSgsgsrg | | detected |
| M5 | gaTANAFLSgsgsrg | Cyclic | 100 |
| M6 | gaIRGILRVVAL | Linear | not |
| | | | detected |
| M7 | gaCKDCSFG | Linear | not |
| | | | detected |
| M8 | gaLPNAAPSLVGS | Cyclic | not |
| | GSRG | | detected |
| M10 | gaGRKKRRQRWRG | Linear | not |
| | AGEQKHPP | | detected |
| M11 | gaLPPSLVQTWVV | Linear | not |
| | VVAL | | detected |
| M14 | gaPVHPQMQTETG | Cyclic | 1200 |
| | TAHCgsgsrg | | |

Tested sequences are shown in capital letters.
Lower case letters represent linker sequences.

### REFERENCES

[1] Hopkins, A. L., Groom, C. R., The druggable genome. Nat. Rev. Drug Discov. 2002, 1, 727-730.
[2] Drews, J., Drug discovery: A historical perspective. Science (80-.). 2000, 287, 1960-1964.
[3] Gorr, S. U., Flory, C. M., Schumacher, R. J., In vivo activity and low toxicity of the second-generation antimicrobial peptide DGL13K. PLoS One 2019, 14, DOI: 10.1371/journal.pone.0216669.
[4] Craik, D. J., Fairlie, D. P., Liras, S., Price, D., The Future of Peptide-based Drugs. Chem. Biol. Drug Des. 2013, 81, 136-147.
[5] Mariani, M., Bracci, L., Presentini, R., Nucci, D., Neri, P., Antoni, G., Immunogenicity of a free synthetic peptide: Carrier-conjugation enhances antibody affinity for the native protein. Mol. Immunol. 1987, 24, 297-303.
[6] Van Regenmortel, M. H. V., Biologicals. Academic Press 2001, pp. 209-213.
[7] Walrant, A., Cardon, S., Burlina, F., Sagan, S., Membrane Crossing and Membranotropic Activity of Cell-Penetrating Peptides: Dangerous Liaisons? Acc. Chem. Res. 2017, 50, 2968-2975.
[8] Dougherty, P. G., Sahni, A., Pei, D., Understanding Cell Penetration of Cyclic Peptides. Chem. Rev. 2019, 119, 10241-10287.
[9] Marasco, D., Perretta, G., Sabatella, M., Ruvo, M., Past and Future Perspectives of Synthetic Peptide Libraries. Curr. Protein Pept. Sci. 2008, 9, 447-467.
[10] Lam, K. S., Salmon, S. E., Hersh, E. M., Hruby, V. J., Kazmierskit, W. M., Knappt, R. J., A new type of synthetic peptide library for identifying ligand-binding activity. Nature 1991, 354, 82-84.
[11] Smith, G. P., Filamentous fusion phage: Novel expression vectors that display cloned antigens on the virion surface. Science (80,). 1985, 228, 1315-1317.
[12] Bowen, J., Schneible, J., Bacon, K., Labar, C., Menegatti, S., Rao, B. M., Screening of yeast display libraries of enzymatically treated peptides to discover macrocyclic peptide ligands. Int. J. Mol. Sci. 2021, 22, 1-20.
[13] Nemoto, N., Miyamoto-Sato, E., Husimi, Y., Yanagawa, H., In vitro virus: Bonding of mRNA bearing puromycin at the 3′-terminal end to the C-terminal end of its encoded protein on the ribosome in vitro. FEBS Lett. 1997, 414, 405-408.
[14] Roberts, R. W., Szostak, J. W., RNA-peptide fusions for the in vitro selection of peptides and proteins. Proc. Natl. Acad. Sci. U.S.A. 1997, 94, 12297-12302.
[15] Tavassoli, A., Benkovic, S. J., Split-intein mediated circular ligation used in the synthesis of cyclic peptide libraries in E. coli. Nat. Protoc. 2007, 2, 1126-1133.
[16] Tavassoli, A., Lu, Q., Gam, J., Pan, H., Benkovic, S. J., Cohen, S. N., Inhibition of HIV budding by a genetically selected cyclic peptide targeting the Gag-TSG101 interaction. ACS Chem. Biol. 2008, 3, 757-764.
[17] Bhagawati, M., Terhorst, T. M. E., Füsser, F., Hoffmann, S., Pasch, T., Pietrokovski, S., Mootz, H. D., A mesophilic cysteine-less split intein for protein trans-splicing applications under oxidizing conditions. Proc. Natl. Acad. Sci. U.S.A. 2019, 116, 22164-22172.
[18] Aranko, A. S., Wlodawer, A., Iwaï, H., Nature's recipe for splitting inteins. Protein Eng. Des. Sel. 2014, 27, 263-271.
[19] Ferraro, D. M., Hope, E. K., Robertson, A. D., Site-specific reflex response of ubiquitin to loop insertions. J. Mol. Biol. 2005, 352, 575-584.
[20] Goyal, B., Goyal, D., Targeting the Dimerization of the Main Protease of Coronaviruses: A Potential Broad-Spectrum Therapeutic Strategy. ACS Comb. Sci. 2020, 22, 297-305.
[21] Ziebuhr, J., The coronavirus replicase. Curr. Top. Microbiol. Immunol. 2005, 287, 57-94.
[22] Anand, K., Ziebuhr, J., Wadhwani, P., Mesters, J. R., Hilgenfeld, R., Coronavirus main proteinase (3CLpro) Structure: Basis for design of anti-SARS drugs. Science (80-.). 2003, 300, 1763-1767.
[23] Baker, R. T., Protein expression using ubiquitin fusion and cleavage. Curr. Opin. Biotechnol. 1996, 7, 541-546.
[24] Yoo, Y., Rote, K., Rechsteiner, M., Synthesis of peptides as cloned ubiquitin extensions. J. Biol. Chem. 1989, 264, 17078-17083.
[25] Muramatsu, T., Takemoto, C., Kim, Y. T., Wang, H., Nishii, W., Terada, T., Shirouzu, M., Yokoyama, S., SARS-CoV 3CL protease cleaves its C-terminal autoprocessing site by novel subsite cooperativity. Proc. Natl. Acad. Sci. U.S.A. 2016, 113, 12997-13002.
[26] Jiang, P., Mu, S., Li, H., Li, Y., Feng, C., Jin, J. M., Tang, S. Y., Design and application of a novel high-throughput screening technique for 1-deoxynojirimycin. Sci. Rep. 2015, 5, DOI: 10.1038/srep08563.
[27] McLoughlin, S. Y., Jackson, C., Liu, J. W., Ollis, D., Increased expression of a bacterial phosphotriesterase in Escherichia coli through directed evolution. Protein Expr. Purif. 2005, 41, 433-440.
[28] Boersma, Y. L., Dröge, M. J., van der Sloot, A. M., Pijning, T., Cool, R. H., Dijkstra, B. W., Quax, W. J., A novel genetic selection system for improved enantioselectivity of Bacillus subtilis lipase A. ChemBioChem 2008, 9, 1110-1115.
[29] Dickinson, B. C., Packer, M. S., Badran, A. H., Liu, D. R., A system for the continuous directed evolution of proteases rapidly reveals drug-resistance mutations. Nat. Commun. 2014, 5, DOI: 10.1038/ncomms6352.
[30] Shie, J. J., Fang, J. M., Kuo, T. H., Kuo, C. J., Liang, P. H., Huang, H. J., Wu, Y. T., Jan, J. T., Cheng, Y. S. E., Wong, C. H., Inhibition of the severe acute respiratory syndrome 3CL protease by peptidomimetic α,β-unsaturated esters. Bioorganic Med. Chem. 2005, 13, 5240-5252.
[31] Jenwitheesuk, E., Samudrala, R., Identifying inhibitors of the SARS coronavirus proteinase. Bioorganic Med. Chem. Lett. 2003, 13, 3989-3992.
[32] Kang, C. B., Gayen, S., Wang, W., Severin, R., Chen, A. S., Lim, H. A., Chia, C. S. B., Schüller, A., Doan, D. N. P., Poulsen, A., Hill, J., Vasudevan, S. G., Keller, T. H., Exploring the binding of peptidic West Nile virus NS2B-NS3 protease inhibitors by NMR. Antiviral Res. 2013, 97, 137-144.
[33] Yang, H., Xie, W., Xue, X., Yang, K., Ma, J., Liang, W., Zhao, Q., Zhou, Z., Pei, D., Ziebuhr, J., Hilgenfeld, R., Kwok, Y. Y., Wong, L., Gao, G., Chen, S., Chen, Z., Ma, D., Bartlam, M., Rao, Z., Design of wide-spectrum inhibitors targeting coronavirus main proteases. PLoS Biol. 2005, 3, DOI: 10.1371/journal.pbio.0030324.
[34] Pillaiyar, T., Manickam, M., Namasivayam, V., Hayashi, Y., Jung, S. H., An overview of severe acute respiratory syndrome-coronavirus (SARS-CoV) 3CL protease inhibitors: Peptidomimetics and small molecule chemotherapy. J. Med. Chem. 2016, 59, 6595-6628.
[1] Hopkins, A. L., Groom, C. R., The druggable genome. Nat. Rev. Drug Discov. 2002, 1, 727-730.
[2] Drews, J., Drug discovery: A historical perspective. Science (80-.). 2000, 287, 1960-1964.
[3] Gorr, S. U., Flory, C. M., Schumacher, R. J., In vivo activity and low toxicity of the second-generation antimicrobial peptide DGL13K. PLoS One 2019, 14, DOI: 10.1371/journal.pone.0216669.
[4] Craik, D. J., Fairlie, D. P., Liras, S., Price, D., The Future of Peptide-based Drugs. Chem. Biol. Drug Des. 2013, 81, 136-147.
[5] Mariani, M., Bracci, L., Presentini, R., Nucci, D., Neri, P., Antoni, G., Immunogenicity of a free synthetic peptide: Carrier-conjugation enhances antibody affinity for the native protein. Mol. Immunol. 1987, 24, 297-303.
[6] Van Regenmortel, M. H. V., Biologicals. Academic Press 2001, pp. 209-213.
[7] Walrant, A., Cardon, S., Burlina, F., Sagan, S., Membrane Crossing and Membranotropic Activity of Cell-Penetrating Peptides: Dangerous Liaisons? Acc. Chem. Res. 2017, 50, 2968-2975.
[8] Dougherty, P. G., Sahni, A., Pei, D., Understanding Cell Penetration of Cyclic Peptides. Chem. Rev. 2019, 119, 10241-10287.
[9] Marasco, D., Perretta, G., Sabatella, M., Ruvo, M., Past and Future Perspectives of Synthetic Peptide Libraries. Curr. Protein Pept. Sci. 2008, 9, 447-467.
[10] Lam, K. S., Salmon, S. E., Hersh, E. M., Hruby, V. J., Kazmierskit, W. M., Knappt, R. J., A new type of synthetic peptide library for identifying ligand-binding activity. Nature 1991, 354, 82-84.
[11] Smith, G. P., Filamentous fusion phage: Novel expression vectors that display cloned antigens on the virion surface. Science (80-.). 1985, 228, 1315-1317.
[12] Bowen, J., Schneible, J., Bacon, K., Labar, C., Menegatti, S., Rao, B. M., Screening of yeast display libraries of enzymatically treated peptides to discover macrocyclic peptide ligands. Int. J. Mol. Sci. 2021, 22, 1-20.
[13] Nemoto, N., Miyamoto-Sato, E., Husimi, Y., Yanagawa, H., In vitro virus: Bonding of mRNA bearing puromycin at the 3′-terminal end to the C-terminal end of its encoded protein on the ribosome in vitro. FEBS Lett. 1997, 414, 405-408.
[14] Roberts, R. W., Szostak, J. W., RNA-peptide fusions for the in vitro selection of peptides and proteins. Proc. Natl. Acad. Sci. U.S.A. 1997, 94, 12297-12302.
[15] Tavassoli, A., Benkovic, S. J., Split-intein mediated circular ligation used in the synthesis of cyclic peptide libraries in E. coli. Nat. Protoc. 2007, 2, 1126-1133.
[16] Tavassoli, A., Lu, Q., Gam, J., Pan, H., Benkovic, S. J., Cohen, S. N., Inhibition of HIV budding by a genetically selected cyclic peptide targeting the Gag-TSG101 interaction. ACS Chem. Biol. 2008, 3, 757-764.
[17] Bhagawati, M., Terhorst, T. M. E., Füsser, F., Hoffmann, S., Pasch, T., Pietrokovski, S., Mootz, H. D., A mesophilic cysteine-less split intein for protein trans-splicing applications under oxidizing conditions. Proc. Natl. Acad. Sci. U.S.A. 2019, 116, 22164-22172.
[18] Aranko, A. S., Wlodawer, A., Iwaï, H., Nature's recipe for splitting inteins. Protein Eng. Des. Sel. 2014, 27, 263-271.
[19] Ferraro, D. M., Hope, E. K., Robertson, A. D., Site-specific reflex response of ubiquitin to loop insertions. J. Mol. Biol. 2005, 352, 575-584.
[20] Goyal, B., Goyal, D., Targeting the Dimerization of the Main Protease of Coronaviruses: A Potential Broad-Spectrum Therapeutic Strategy. ACS Comb. Sci. 2020, 22, 297-305.
[21] Ziebuhr, J., The coronavirus replicase. Curr. Top. Microbiol. Immunol. 2005, 287, 57-94.
[22] Anand, K., Ziebuhr, J., Wadhwani, P., Mesters, J. R., Hilgenfeld, R., Coronavirus main proteinase (3CLpro) Structure: Basis for design of anti-SARS drugs. Science (80-.). 2003, 300, 1763-1767.
[23] Baker, R. T., Protein expression using ubiquitin fusion and cleavage. Curr. Opin. Biotechnol. 1996, 7, 541-546.
[24] Yoo, Y., Rote, K., Rechsteiner, M., Synthesis of peptides as cloned ubiquitin extensions. J. Biol. Chem. 1989, 264, 17078-17083.
[25] Muramatsu, T., Takemoto, C., Kim, Y. T., Wang, H., Nishii, W., Terada, T., Shirouzu, M., Yokoyama, S., SARS-CoV 3CL protease cleaves its C-terminal autoprocessing site by novel subsite cooperativity. Proc. Natl. Acad. Sci. U.S.A. 2016, 113, 12997-13002.
[26] Jiang, P., Mu, S., Li, H., Li, Y., Feng, C., Jin, J. M., Tang, S. Y., Design and application of a novel high-throughput screening technique for 1-deoxynojirimycin. Sci. Rep. 2015, 5, DOI: 10.1038/srep08563.
[27] McLoughlin, S. Y., Jackson, C., Liu, J. W., Ollis, D., Increased expression of a bacterial phosphotriesterase in Escherichia coli through directed evolution. Protein Expr. Purif. 2005, 41, 433-440.
[28] Boersma, Y. L., Dröge, M. J., van der Sloot, A. M., Pijning, T., Cool, R. H., Dijkstra, B. W., Quax, W. J., A novel genetic selection system for improved enantioselectivity of Bacillus subtilis lipase A. ChemBioChem 2008, 9, 1110-1115.
[29] Dickinson, B. C., Packer, M. S., Badran, A. H., Liu, D. R., A system for the continuous directed evolution of proteases rapidly reveals drug-resistance mutations. Nat. Commun. 2014, 5, DOI: 10.1038/ncomms6352.
[30] Shie, J. J., Fang, J. M., Kuo, T. H., Kuo, C. J., Liang, P. H., Huang, H. J., Wu, Y. T., Jan, J. T., Cheng, Y. S. E., Wong, C. H., Inhibition of the severe acute respiratory syndrome 3CL protease by peptidomimetic α,β-unsaturated esters. Bioorganic Med. Chem. 2005, 13, 5240-5252.
[31] Jenwitheesuk, E., Samudrala, R., Identifying inhibitors of the SARS coronavirus proteinase. Bioorganic Med. Chem. Lett. 2003, 13, 3989-3992.
[32] Kang, C. B., Gayen, S., Wang, W., Severin, R., Chen, A. S., Lim, H. A., Chia, C. S. B., Schüller, A., Doan, D. N. P., Poulsen, A., Hill, J., Vasudevan, S. G., Keller, T. H., Exploring the binding of peptidic West Nile virus NS2B-NS3 protease inhibitors by NMR. Antiviral Res. 2013, 97, 137-144.
[33] Yang, H., Xie, W., Xue, X., Yang, K., Ma, J., Liang, W., Zhao, Q., Zhou, Z., Pei, D., Ziebuhr, J., Hilgenfeld, R., Kwok, Y. Y., Wong, L., Gao, G., Chen, S., Chen, Z., Ma, D., Bartlam, M., Rao, Z., Design of wide-spectrum inhibitors targeting coronavirus main proteases. PLoS Biol. 2005, 3, DOI: 10.1371/journal.pbio.0030324.
[34] Pillaiyar, T., Manickam, M., Namasivayam, V., Hayashi, Y., Jung, S. H., An overview of severe acute respiratory syndrome-coronavirus (SARS-CoV) 3CL protease inhibitors: Peptidomimetics and small molecule chemotherapy. J. Med. Chem. 2016, 59, 6595-6628.

What is claimed is:

1. A Method for selecting peptide inhibitors based on protein cytotoxicity, comprising:
1) inserting a cytotoxic target protein and a library of peptide variants in a host cell;
2) expressing the cytotoxic protein and peptide variants in the host cell; and
3) identifying the peptide variants that block the cytotoxic target protein.

2. The method of claim 1, wherein the cytotoxic target protein is formed by synthesizing a cytotoxic protein gene and cloning the cytotoxic protein gene with an expression vector in a host cell.

3. The method of claim 1, wherein toxicity of the cytotoxic target protein is validated by expressing the cytotoxic protein in the host cell.

4. The method of claim 1, wherein the library of peptide variants is formed by synthesizing a peptide library or building a peptide library using degenerate oligos and by cloning the peptide library with an expression vector in the host cell.

5. The method of claim 1, wherein the cytotoxic target protein and peptide are expressed by adding an inducer to the host cell.

6. The method of claim 1, wherein the peptide variants are identified by identifying growing clones through growth on solid media or in a liquid culture and by identifying peptide sequences of the peptides that inhibit the cytototoxic target protein through sanger or Next Generation Sequencing

7. The method of claim 1, wherein the in-vitro inhibitory potency of the peptides on the cytotoxic target protein is tested.

8. A Method for selecting peptide inhibitors based on protein cytotoxicity, comprising:
1) inserting a cytotoxic target protein and a library of peptide variants in a host cell;
2) expressing the cytotoxic protein and peptide variants in the host cell; and
3) identifying the peptide variants that block the cytotoxic target protein,
wherein the cytotoxic target protein is formed by synthesizing a cytotoxic protein gene and cloning the cytotoxic protein gene with an expression vector in a host cell, and
wherein the library of peptide variants is formed by synthesizing a peptide library or building a peptide library using degenerate oligos and by cloning the peptide library with an expression vector in the host cell.

9. The method of claim 8, wherein toxicity of the cytotoxic target protein is validated by expressing the cytotoxic protein in the host cell.

10. The method of claim 8, wherein the cytotoxic target protein and peptide are expressed by adding an inducer to the host cell.

11. The method of claim 8, wherein the peptide variants are identified by identifying growing clones through growth on solid media or in a liquid culture and by identifying peptide sequences of the peptides that inhibit the cytototoxic target protein through sanger or Next Generation Sequencing

12. The method of claim 8, wherein the in-vitro inhibitory potency of the peptides on the cytotoxic target protein is tested.

13. A Method for selecting peptide inhibitors based on protein cytotoxicity, comprising:
1) inserting a cytotoxic target protein and a library of peptide variants in a host cell;
2) expressing the cytotoxic protein and peptide variants in the host cell; and
3) identifying the peptide variants that block the cytotoxic target protein,
wherein toxicity of the cytotoxic target protein is validated by expressing the cytotoxic protein in the host cell,
and wherein the cytotoxic target protein and peptide are expressed by adding an inducer to the host cell.

14. The method of claim 13, wherein the cytotoxic target protein is formed by synthesizing a cytotoxic protein gene and cloning the cytotoxic protein gene with an expression vector in a host cell.

15. The method of claim 13, wherein the library of peptide variants is formed by synthesizing a peptide library or building a peptide library using degenerate oligos and by cloning the peptide library with an expression vector in the host cell.

16. The method of claim 13, wherein the peptide variants are identified by identifying growing clones through growth on solid media or in a liquid culture and by identifying peptide sequences of the peptides that inhibit the cytototoxic target protein through sanger or Next Generation Sequencing

17. The method of claim 13, wherein the in-vitro inhibitory potency of the peptides on the cytotoxic target protein is tested.
